(19) United States
(12) Patent Application Publication
(10) Pub. No.: US 2008/0114306 A1
(43) Pub. Date: May 15, 2008

(54) Small volume syringe with writing portion

(76) Inventor: Rex O. Bare, Lake Forest, CA (US)

Correspondence Address: STETINA BRUNDA GARRED & BRUCKER, 75 ENTERPRISE, SUITE 250, ALISO VIEJO, CA, 92656, US

(21) Appl. No.: 11/981,000

(22) Filed: Oct. 29, 2007

Related U.S. Application Data
(60) Provisional application No. 60/856,575, filed on Nov. 3, 2006.

(51) Int. Cl. A61M 5/00; A61M 5/315

(52) U.S. Cl. 604/218; 604/187

(57) ABSTRACT

A small volume safety syringe is provided herein. A rigid shell or syringe body of the small volume safety syringe may define an exterior surface. A writing surface which is sufficiently large and flat for a medical professional to write medically pertinent information on the writing surface may be formed on the exterior surface of the small volume safety syringe. Moreover, a white pigment may be disposed on the writing surface so as to make the written information more visible and readable.

### CROSS-REFERENCE TO RELATED APPLICATIONS

This application claims the benefit of U.S. Provisional Patent Application Ser. No. 60/856,575, filed Nov. 3, 2006, entitled Small Volume Syringe with Writing Surface, the entire contents of which is expressly incorporated herein by reference.

This application further is related to U.S. Provisional Patent Application Ser. No. 60/793,022, filed Apr. 19, 2006, entitled Vacuum Actuated Small Volume Syringe, the entire content of which is incorporated herein by reference.

### STATEMENT RE: FEDERALLY SPONSORED RESEARCH/DEVELOPMENT

Not Applicable

### BACKGROUND

The present invention relates to a small volume syringe with a writing portion.

Syringes come in many sizes and styles. For examples, syringes may be provided to medical professionals or users in sizes of 1 cc, 2 cc, 3 cc, etc. For larger volume syringes, the outer surface has a surface that is sufficiently large and flat such that the medical professional or user may write directly upon the outer surface to indicate various information such as the person's name who should be injected, expiration date of the fluidic medication, the name of the fluidic medication, etc. Typically, the “writing surface” is defined by the body of the syringe, specifically, the outer surface of the syringe body.

Unfortunately, for smaller volume syringes (e.g., 1 cc or less). The outer surface of the syringe body is too small for the medical professional to write upon. By way of example and not limitation, the outer surface of the syringe body may be (1) too round for the medical professional or user to write upon or (2) too short. Such small outer diameter does not provide a sufficient flat area for the medical professional or user to write upon to indicate various information pertinent to the small volume syringe. Also, the length of the outer surface of the syringe body may be too short for the medical professional or user to write upon.

To write information on small volume syringes, the medical professional or user must write on a separate label with pressure adhesive backing then adhere the label to the outer surface of the syringe body. Unfortunately, writing on a separate label then adhering the label to the small volume syringe is cumbersome. The medical professional must simultaneously handle both the syringe and the label having the pressure adhesive backing. The pressure adhesive backing may sometimes adhere inconveniently to the fingers of the medical professional as well as the small volume syringe in an incorrect orientation. In such situation, the medical professional must release the adhesive, re-orient the label and re-attach the label to the small volume syringe. The medical professional must manipulate the safety syringe with the exposed needle and the label with pressure adhesive backing simultaneously. Due to the difficulty of working with the pressure adhesive backing, the medical professional may accidentally prick himself/herself with the needle of the small volume syringe.

Another disadvantage or deficiency in writing pertinent medical information on a separate label is that the wrong label may be associated or adhered to the small volume syringe. By way of example and not limitation, the medical professional or user may be handling a plurality of syringes and labels simultaneously. In this situation, it would be easy for the medical professional or user to mistakenly adhere the wrong label to the syringe.

### BRIEF SUMMARY

The small volume safety syringe discussed herein addresses the deficiencies identified above, below and those that are known in the art.

The small volume safety syringe discussed herein may be a 1 cc or less small volume syringe. Typically, in prior art small volume safety syringes, the outer diameter of the syringe body of the small volume safety syringe may be too small or the length of the syringe body may be too short for a medical professional or user to write pertinent medical information regarding the safety syringe, fluidic medication or patient as well as other information that should be written on the safety syringe, as discussed above.

To allow the medical professional or user to write pertinent medical information on the small volume safety syringe, an exterior surface of the small volume safety syringe may be formed with a writing surface. The writing surface may be sufficiently large and flat so as to allow the medical professional or user to write pertinent medical information on the writing surface and thereby associate pertinent medical information with the safety syringe.

A white pigment may be disposed on the writing surface via a screen printing process or any other process known in the art. The white pigment provides a distinguishable background such that the information written on the writing surface may be easily readable and visible. It is also contemplated that other pigment colors may be disposed on the writing surface. For example, black, yellow, red, etc.

For a standard small volume safety syringe (e.g., non-retractable safety syringe), the exterior surface may be defined by the syringe body of the small volume safety syringe. More particularly, a small volume safety syringe may comprise a needle which is engaged to a distal portion of the syringe body. The syringe may also comprise a piston, shaft and thumb platform wherein the piston and a distal portion of the shaft are disposed within the syringe body and a proximal portion of the shaft and the thumb platform are disposed external to the syringe body.

In use, the piston and the shaft is slideably traversable within the syringe body. The outer surface of the syringe body is continuously exposed to the environment or viewable by the medical professional or user throughout operation of the syringe. Accordingly, the outer surface of the syringe body may be characterized as the exterior surface of the small volume safety syringe.

The writing surface formed on the syringe body may be integrally formed with the syringe body. By way of example and not limitation, the syringe body may be fabricated via a plastic injection molding process. The writing surface integrally formed with syringe body may have a width about equal to the outer diameter of the syringe body. Plastic material may be added to the syringe body to build up one side of the syringe body to form the writing surface. It is also contemplated that the syringe body may be formed with two or more writing surfaces. For example, optionally, the opposed side of the syringe body may be formed with a second writing surface to increase the amount of information that may be associated with the syringe.

It is also contemplated that a width of the writing surface may be less than an outer diameter of the syringe body. Also, it is contemplated that a width of the writing surface may be greater than an outer diameter of the syringe body. For example, the width of the writing surface may be varied up to about three (3) times the outer diameter of the syringe body.

In another example, an exterior surface of the small volume safety syringe may be defined by an outer surface of a rigid shell. For example, the small volume safety syringe disclosed in U.S. Patent Application Ser. No. 60/793,022 (hereinafter the '022 application) may comprise an internal vacuum compartment as well as an external vacuum compartment. The external vacuum compartment may be defined by 1) a syringe body, 2) a shell which is slidable over the syringe body, 3) distal seal and 4) proximal seal. During use of the retractable safety syringe disclosed in the '022 application, the shell slides over (i.e. covers) the outer surface of the syringe body. As such, the medical professional may not be able to see the outer surface of the syringe body during certain steps of operating the small volume safety syringe. In these situations, if the writing surface were to be formed on the outer surface of the syringe body, as discussed above, the medical professional may not be able to read the pertinent medical information on the writing surface because the shell may cover the writing surface. Fortunately, the outer surface of the rigid shell is always or continuously viewable by the medical professional or user throughout operation of the syringe. In this respect, the exterior surface of the safety syringe disclosed in the '022 application may be defined by the outer surface of the rigid shell. The writing surface may be integrally formed with the rigid shell with a width of the writing surface greater than, equal to or less than a diameter of the shell. The writing surface and rigid shell may be formed via an injection molding process.

It is also contemplated that a second writing surface may be integrally formed with the rigid shell opposite the first writing surface.

Optionally, a pigment (e.g., white, red, blue, etc.) may be disposed on the writing surface to make information written on the writing surface readable and viewable.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a front view of a syringe;

FIG. 2 is a bottom cross-sectional view of the syringe shown in FIG. 1 illustrating a first writing surface of a first embodiment integrally formed with a syringe body;

FIG. 2A is a second embodiment of the first writing surface integrally formed with the syringe body wherein a width of the writing surface is less than an outer diameter of the syringe body;

FIG. 2B is a third embodiment of the first writing surface wherein a width of the first writing surface is greater than outer diameter of the syringe body;

FIG. 3 is a perspective view of a small volume retractable safety syringe;

FIG. 4 is a cross sectional perspective view of the small volume retractable safety syringe shown in FIG. 3;

FIG. 5 is a bottom cross sectional view of the syringe shown in FIG. 3 illustrating a first embodiment of a first writing surface integrally formed with a syringe body;

FIG. 5A is an illustration of a second embodiment of the first writing surface wherein a width of the first writing surface is less than an outer diameter of the syringe body; and

FIG. 5B is an illustration of the third embodiment of the first writing surface wherein a width of the first writing surface is greater than an outer diameter of the syringe body.

### DETAILED DESCRIPTION

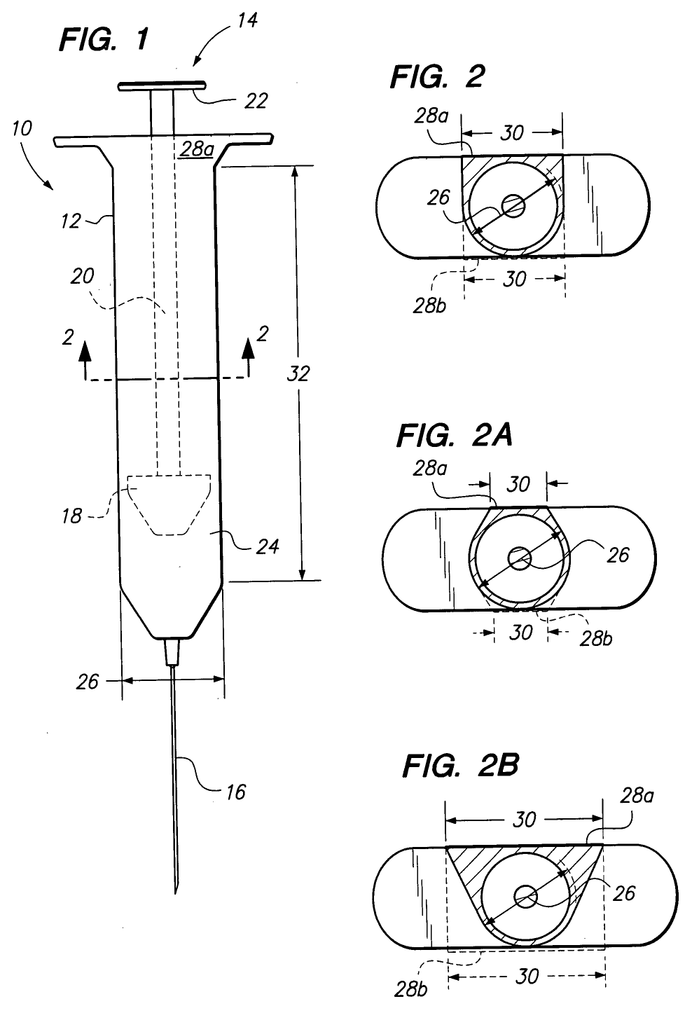

Referring now to the drawings which are for the purpose of illustrating preferred embodiments of the syringe 10 and not for the purpose of limiting the same, FIG. 1 illustrates a small volume syringe 10. The syringe 10 may comprise a syringe body 12, a plunger 14, and a needle 16. The needle 16 may be engaged to a distal portion of the syringe body 12. The plunger 14 may comprise a piston 18, shaft 20 and a thumb platform 22. The shaft 20 may be attached to the piston 18 and the thumb platform 22. The piston 18 and a distal portion of the shaft 20 may be disposed within the syringe body 12. The thumb platform 22 and a proximal portion of the shaft 20 may be disposed outside of the body. The thumb platform 22 may be traversed closer to or further away from the syringe body 12 to traverse the piston 18 within the body. The piston 18, body 12 and needle 16 define a variable fluid chamber 24. For small volume syringes 10, the variable fluid chamber 24 may have a volume of about 1 cc or less and the syringe body 12 may define an outer diameter 26.

The body of the syringe 10 may have a first writing surface 28a (see FIGS. 1 and 2) which is sufficiently large and flat such that a medical professional or user may write pertinent medical information on the first writing surface 28a. A width 30 of the first writing surface 28a may be about equal to, greater than or less than the outer diameter 26 of the syringe body 12, as shown in FIGS. 2, 2A and 2B. Also, a length 32 of the writing surface may extend partially or entirely along the length of the syringe body 12. Preferably, the length 32 and width 30 of the writing surface may be sufficient for the medical professional or user to write pertinent medical information regarding the small volume syringe 10 or the fluidic medication to be injected into the patient.

The first writing surface 28a may be integrally formed with the syringe body 12. For example, additional material may be added to the syringe body 12 to provide for the sufficiently large and flat writing surface as shown in FIGS. 2, 2A, and 2B. In particular, the first writing surface 28a and syringe body 12 may fabricated via an injection molding process.

It is also contemplated that a second writing surface 28b may be integrally formed with the syringe body 12, as shown by the dashed lines in FIGS. 2, 2A, and 2B. In particular, the first and second writing surfaces 28a, 28b may be on opposite sides of the syringe body 12. The first and second writing surfaces 28a, 28b may have a width 30 which is equal to (see FIG. 2), less than (see FIG. 2A), or greater than (see FIG. 2B) an outer diameter 26 of the syringe body 12. It is also contemplated that the width 30 of the first writing surface 28a and the width 30 of the second writing surface 28b may be different. All combinations of widths 30 of the first and second writing surfaces 28a, 28b are contemplated. By way of example and not limitation, the width 30 of the first writing surface 28a may be greater than the width 30 of the outer diameter 26 of the syringe body 12 and the width 30 of the second writing surface 28b may be less than or equal to the outer diameter 26 of the syringe body 12.

A white pigment may be disposed on the first and/or second writing surfaces 28a, 28b such that the information written on the writing surface(s) 28a, 28b may be easily viewable and readable throughout operation of the syringe 10. The white pigment may be disposed on the writing surface(s) 28a, 28b via a screen printing process or any other process known in the art. It is also contemplated that other colors such as black, yellow, red, etc. may be disposed on the writing surface(s) 28a, 28b.

In use, the medical professional may write pertinent medical information on the writing surface(s) 28a, 28b of the small volume syringe 10. For example, the medical professional may write the patient's name, expiration date of the fluidic medication, the brand name as well as the generic name for the fluidic medication, as well as other pertinent medical information related either to the patient, syringe 10, fluidic medication, and other relevant information.

The writing surface 28 may be formed on an exterior surface of other types of small volume syringes 10. For example, one specialty type of small volume syringe is disclosed in U.S. Provisional Patent Application Ser. No. 60/793,022 (hereinafter, the '022 application), the entire content of which is incorporated herein by reference.

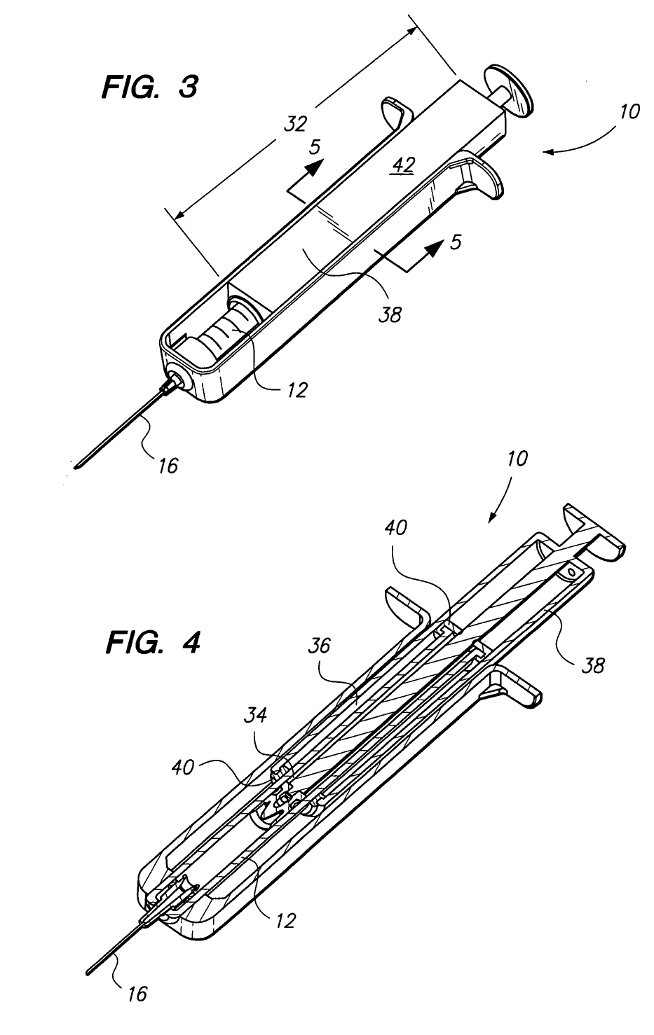

The '002 application discloses a retractable small volume safety syringe 10, as shown in FIGS. 3-5B. The needle 16 is retracted into the syringe body 12 by retraction forces created by an internal variable vacuum compartment 34 (see FIG. 4) and an external variable vacuum compartment 36 (see FIG. 4). The retraction force created by the external vacuum compartment 36 is added to the retraction force of the internal vacuum compartment 34 based on a view that the retraction force of the internal vacuum compartment 34 alone may be insufficient to overcome any frictional forces holding the needle 16 to the distal end of the syringe body 12 of the retractable small volume safety syringe 10. In this retractable small volume safety syringe 10, the external variable vacuum compartment 36 is formed by a rigid shell 38, seals 40 and the syringe body 12. At a retracted position, the rigid shell 38 does not cover the syringe body 12. However, the rigid shell 38 covers the syringe body 12 as the syringe is traversed toward the filling or engaged position, as shown in FIG. 3. Any markings or indicia written on the outer surface of the syringe body 12 will not be visible or readable when the syringe 10 is traversed towards the engaged position or filling position. Accordingly, the outer surface of the syringe body 12 is not visible throughout the entire operation of the small volume retractable safety syringe 10.

Accordingly, the exterior surface on which the writing surfaces 28a, 28b may be formed may be defined by the outer surface 42 of the rigid shell 38. During operation of the syringe 10, the outer surface 42 of the rigid shell 38 is always visible to the medical professional or user. As such, the first and/or second writing surface(s) 28a, 28b may be formed on the outer surface 42 of the rigid shell 38.

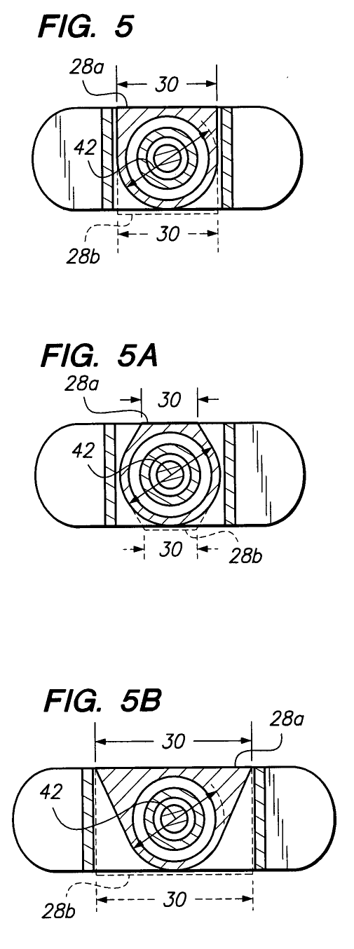

The first and second writing surfaces 28a, 28b formed on the outer surface 42 of the rigid shell 38 may have a width which is equal to (see FIG. 5), less than (see FIG. 5A), or greater than (see FIG. 5B) an outer diameter 42 of the rigid shell 38. It is also contemplated that the width 30 of the first writing surface 28a and the width 30 of the second writing surface 28b may be different. All combinations of widths 30 of the first and second writing surfaces 28a, 28b are contemplated. By way of example and not limitation, the width 30 of the first writing surface 28a may be greater than the width of the outer diameter 42 of the rigid shell 38 and the width 30 of the second writing surface 28b may be less than or equal to the outer diameter 42 of the rigid shell 38. Moreover, a length 32 of the writing surface(s) 28a, 28b may extend partially or entirely along the length of the rigid shell 38.

A white pigment may be disposed on the writing surface(s) 28a, 28b so as to allow the medical professional or user to write on the writing surface(s) 28a, 28b with a black colored marker. In this way, the pertinent medical information is more readily readable and visible. The white pigment may be disposed on the writing surface(s) 28a, 28b via a screen printing process or any other process known in the art. It is also contemplated that other colors may be disposed on the writing surface(s) 28a, 28b such as black, yellow, red, etc.

Although the writing surface(s) 28a, 28b was described in relation to a safety syringe 10 for injecting a patient with fluidic medication, it is also contemplated that the writing surface 28a, 28b may be formed on other types of safety syringes 10 such as those that serve to withdraw fluid from a container or bodily fluids.

As used herein, the retracted position of the syringe 10 is where the piston 18 is distanced from the needle 16. Typically, the piston 18 is closely adjacent to the proximal end of the syringe body 12. The filling position of the syringe 10 refers to the syringe 10 before fluidic medication has been filled into the syringe 10. The piston 18 is closer to the distal end of the syringe body 12 than to the proximal end of the syringe body 12. More particularly, the piston 18 is closely adjacent to the distal end of the syringe body 12. The engaged position of the syringe 10 refers to the syringe 10 after fluidic medication has been filled into the variable fluid chamber 24, the fluidic medication has been injected into the patient, and the piston 18 engages the needle 16 such that subsequent retraction of the piston 18 toward the proximal end of the syringe body 12 retracts the needle 16 into the syringe body 12 to prevent accidental needle 16 pricking. At the engaged position, the piston 18 is at the distal portion of the syringe body 12 and engaged to the needle.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A small volume safety syringe comprising:
a needle;
a syringe body with the needle removeably attachable to the syringe body;
a variable fluid chamber in fluid communication with the needle for introducing fluid into or evacuating fluid out of the variable fluid chamber, the needle being selectively retractable into the variable fluid chamber;
a rigid shell slideably disposeably over the syringe body, the rigid shell and the syringe body defining an external variable vacuum compartment capable of producing a retraction force for retracting the needle into the variable fluid chamber, a sufficiently large and flat first writing surface formed on an outer surface of the rigid shell such that a user may write medically pertinent information on the first writing surface.

2. The syringe of claim 1 further comprising a sufficiently large and flat second writing surface formed on the rigid shell opposite from the first writing surface such that a user may write medically pertinent information on the second writing surface.

3. The syringe of claim 1 wherein a width of the writing surface is greater than an outer diameter of the rigid shell.

4. The syringe of claim 3 wherein the width of the writing surface is three (3) times the outer diameter of the rigid shell.

5. The syringe of claim 1 wherein the writing surface has a white background.

6. A small volume safety syringe for injecting fluidic medication into a patient, the syringe comprising:
a. a syringe body defining an inner surface, an outer surface, a proximal portion and a distal portion, the outer surface defining a first writing surface sufficiently large and flat such that a medical professional may write medically pertinent information on the first writing surface;
b. a needle holder engaged to the distal portion of the syringe body;
c. a needle attached to the needle holder;
d. a plunger having a piston, shaft, and a thumb platform, the piston and a distal portion of the shaft being disposed within the syringe body, the thumb platform and a proximal portion of the shaft being disposed external to the syringe body, the shaft protruding out of the syringe body from the proximal portion of the syringe body, the piston being traversable between a retracted position and an engaged position;
e. a distal seal engaged to a distal portion of the shell and forming an airtight seal between the shell and syringe body; and
f. wherein the distal seal, plunger, body and needle holder define a variable fluid chamber for receiving fluidic medication during use of the syringe, and the variable fluid chamber has a volume of about 1 CC or less.

7. The syringe of claim 5 wherein a width of the writing surface is greater than an outer diameter of the syringe body.

8. The syringe of claim 7 wherein the width of the writing surface is three (3) times the outer diameter of the syringe body.

9. The syringe of claim 6 wherein the writing surface has a white background.

10. The syringe of claim 6 further comprising a second writing surface sufficiently large and flat such that a user may write medically pertinent information on the second writing surface.
